(19) United States
(12) Patent Application Publication
(10) Pub. No.: US 2019/0008971 A1
(43) Pub. Date: Jan. 10, 2019

(54) Implantable Scaffolds for Capturing Metastatic Breast Cancer Cells In Vivo

(71) Applicant: THE REGENTS OF THE UNIVERSITY OF MICHIGAN, Ann Arbor, MI (US)

(72) Inventors: Lonnie D. SHEA, Ann Arbor, MI (US); Shreyas S. RAO, Tuscaloosa, AL (US); Samira AZARIN, Woodbury, MN (US); Jacqueline S. JERUSS, Ann Arbor, MI (US); Grace BUSHNELL, Plymouth, MI (US)

(21) Appl. No.: 16/068,004

(22) PCT Filed: Jan. 6, 2017
(86) PCT No.: PCT/US17/12556

§ 371 Date: Jul. 3, 2018

Related U.S. Application Data
(60) Provisional application No. 62/276,097, filed on Jan. 7, 2016.

(51) Int. Cl. A61K 47/34; A61K 9/00; G01N 33/50

(52) U.S. Cl. CPC A61K 47/34; G01N 33/5088; A61K 9/00

(57) ABSTRACT

The present disclosure relates generally to techniques for capturing cancer cells and, more particularly, to techniques for capturing metastatic cancer cells in vivo.

### CROSS-REFERENCE TO RELATED APPLICATION

This application claims the benefit of U.S. Provisional Patent Application Ser. No. 62/276,097, filed Jan. 7, 2016, the disclosure of which is incorporated herein by reference in its entirety.

### STATEMENT REGARDING FEDERALLY SPONSORED RESEARCH OR DEVELOPMENT

This invention was made with government support under CA173745 awarded by the National Institutes of Health. The Government has certain rights in the invention.

### FIELD OF THE DISCLOSURE

The present disclosure relates generally to techniques for capturing cancer cells and, more particularly, to techniques for capturing metastatic cancer cells in vivo.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

Early detection of tumor cells, especially cancer cells, is the goal for optimum diagnosis. Yet, early detection can be challenging, even when the goal is to detect tumor cells before metastatic spread. Indeed, even metastatic spread often goes undetected. Take the example of breast cancer. Oncogenic progression of breast cancer from the primary tumor to distant metastatic sites is the critical event that defines stage IV disease. Yet, currently, metastatic disease is detected through radiologic imaging modalities after the originating disease has become destructive to the host organ. In fact, the striking lack of robust technologies capable of early detection of metastatic events has profoundly limited the development of life-preserving interventions.

To address these limitations, techniques for detecting circulating tumor cells (CTCs) are being pursued, in both the experimental and clinical settings. While promising, the widespread use of CTC capture faces considerable challenges, especially given the high biomarker sensitivity and specificity required to capture a low number of circulating CTCs. Furthermore, CTCs may not represent the population of cells capable of metastasis. Plus, CTCs can circulate for long periods before invading distant organs, meaning that they too can go undetected.

The capacity to identify metastatic cells or foci at the earliest possible time-point may permit the delivery of targeted treatment interventions prior to the compromise of distant organs, potentially translating into prolonged distant metastasis free outcomes. Thus, there is a need for development of technologies to aid in the detection of metastatic events in the nascent setting.

### SUMMARY OF THE INVENTION

The present techniques describe mechanisms for early detection of metastatic cells using an implanted biomaterial scaffold configured to capture such cells. The scaffolds are capable of capturing metastatic cells and, in particular, over a clinically significant period of time, which was previously not available. Scaffolds have been developed that remain functionalized over sufficiently long time frames to allow for a sufficient amount of cell aggregation for detection. In some instances, scaffolds have been designed that remain functionalized long enough to provide targeted treatment sites in vivo, locations where metastatic cells are not merely just detected, but over time are targeted, and provide a specific location for cell resection and possible removal of all metastatic cells.

The effectiveness of these longer lifetime scaffolds, especially for use as targeted treatment cites, relates to Paget's “seed and soil” paradigm which proposes that, prior to colonization by metastatic cells, supportive cells (e.g., fibroblasts, immune cells, endothelial cells), soluble factors, and extracellular matrix (ECM) components establish a microenvironment conducive to tumor cell homing and colonization. The importance of this paradigm is that metastasis to specific organs is not random, but rather is influenced by the properties of the local environment. The use of longer lifetime scaffolds, in vivo, provides sufficient time for these support cells to establish the microenvironment, at the scaffold, to which metastatic cells are attracted.

In some examples, the present techniques provide a micro-porous poly(ε-caprolactone) (PCL) scaffold. Such PCL scaffolds have a greater stability than the micro-porous poly(lactide-co-glycolide) (PLG) biomaterial scaffolds. The PCL scaffolds, in fact, provide such an unexpectedly greater amount of stability that, for the first time, investigation of the dynamic immune response of a subject, as well as other cellular events associated recruitment of metastatic cells, is demonstrated. In some of these examples, cellular events for breast cancer cells are observed. The disclosure contemplates that the scaffolds and methods provided herein are useful for recruitment and/or capture of any cancer cell including, without limitation, breast cancer, pancreatic cancer, lung cancer, liver cancer, and brain cancer.

In some examples, the present invention provides biomaterial PCL scaffolds and implants derived therefrom to provide a target used to recruit and detect metastatic cells. In some examples, that target is used in vitro, in vivo, in situ, etc. In some examples, the present invention provides a biomaterial PCL scaffold used to capture metastatic cells and allow those cells to colonize at a metastatic site over time.

In accordance with an example, a biomaterial implant provided herein comprises a micro-porous scaffold comprising poly(ε-caprolactone) (PCL) or a poly(ethylene glycol) (PEG) hydrogel and configured to recruit circulating metastatic cells. In further aspects, the present disclosure provides an alginate scaffold.

In accordance with some examples, the scaffold is a PCL scaffold that is characterized by a degradation profile that is a percent degradation over time, and wherein the scaffold has a degradation profile value of less than 50%, 25%, 5%, or 1% degradation over at least 90 days. In further embodiments, the scaffold has a degradation profile value of less than 50%, 25%, 5%, or 1% degradation over at least 6 months, 1 year, 18 months, 2 years, or more.

In accordance with some examples, the biomaterial implant comprises a scaffold comprising PEG and is non-biodegradable and is non-resorbable. In some examples, the PEG scaffold is crosslinked with a peptide or polysaccharide that is not degraded by a mammalian enzyme. In further examples, the PEG scaffold is degraded when the scaffold is contacted with an enzyme found in a prokaryotic cell and said degradation releases recruited and/or captured cells.

Accordingly, the disclosure provides a biomaterial implant comprising a micro-porous scaffold comprising poly(ε-caprolactone) (PCL) or poly(ethylene glycol) (PEG) and configured to recruit circulating metastatic cells. In some embodiments, the scaffold comprises PCL (PCL scaffold) or PEG (PEG scaffold) and is characterized by a degradation profile that is a percent degradation over time, and wherein the scaffold has a degradation profile value of less than 50% degradation over 90 days.

In accordance with some examples, the PCL or PEG scaffold has a degradation profile value that is less than 25% degradation over 90 days. In some examples, the PCL or PEG scaffold has a degradation profile value that is less than 10% degradation over 90 days. In further examples, the PCL or PEG scaffold has a degradation profile value that is less than 5% degradation over 90 days. In some examples, the PCL or PEG scaffold has a degradation profile value that is less than 1% degradation over 90 days.

In various examples, the scaffold comprises PEG (PEG scaffold) and is non-biodegradable and is non-resorbable.

In some examples, the PEG scaffold is crosslinked with a peptide or polysaccharide that is not degraded by mammalian cells to release the recruited circulating metastatic cells. In further examples, the implant has an average mesh size of from about 20 nanometers (nm) to about 50 nm. In some examples, the scaffold is functionalized with at least one of a stromal cell, an extracellular matrix molecule, or a cytokine.

In some examples, the PEG has an average molecular weight of at least 10,000 daltons. In further examples, the PEG has an average molecular weight of at least 15,000 daltons. In still further examples, the PEG has an average molecular weight between about 10,000 and about 20,000 daltons.

In some aspects, the disclosure provides a biomaterial implant comprising a micro-porous scaffold comprising a non-biodegradable polymer configured to recruit circulating metastatic cells and functionalized to release the recruited circulating metastatic cells in response to contact with an external enzyme.

In some aspects, a biomaterial implant is provided comprising a micro-porous scaffold comprising a non-biodegradable polymer configured to recruit circulating metastatic cells and functionalized to degrade in response to contact with an external enzyme to release the recruited circulating metastatic cells.

In accordance with some examples, a method of capturing a metastatic tumor cell is provided comprising implanting the biomaterial implant of the disclosure into a subject. In some examples, the subject suffers from cancer that has been diagnosed as metastatic. In further examples, the subject suffers from cancer that has not been diagnosed as metastatic. In still further examples, the implanting is subcutaneous or intramuscular. In some examples, the implanting occurs at one site in the subject. In some examples, the capturing lowers tumor burden of the subject.

In some examples, the implanting occurs at more than one site in the subject. In further examples, one biomaterial implant is implanted, while in still further examples, more than one biomaterial implant is implanted. In accordance with some examples, the site is the lung, liver, brain, bone, peritoneum, omental fat, muscle, or lymph node.

According to some examples, methods of the disclosure further comprise removing the biomaterial implant or implants. In some examples, methods of the disclosure further comprise detecting a metastatic cell, the detecting comprising one or more of inverse-scattering optical coherence tomography (ISOCT), fluorescence activated cell sorting (FACS), high frequency ultrasound, ultrasound, positron emission tomography (PET) scan, magnetic resonance imaging (MRI), photoacoustic imaging, or fluorescence imaging.

According to some examples, methods of the disclosure further comprise administering to the subject a chemotherapeutic agent. In further examples, methods of the disclosure further comprise surgically removing the cancer from the subject. In some examples, methods of the disclosure further comprise administering radiotherapy to the subject. In still further examples, methods of the disclosure further comprise retrieving the captured metastatic tumor cell from the scaffold. In some examples, methods of the disclosure further comprise retrieving a captured non-tumor cell from the scaffold.

In some examples, survival rate of the subject is increased relative to a subject in whom the biomaterial implant was not implanted.

In accordance with some examples, a method of analyzing effectiveness of a treatment to reduce metastasis in a subject is provided, comprising (i) implanting at least a first and a second biomaterial implant into the subject and maintaining for a period of time wherein each implant is according to an implant described herein; (ii) removing the first biomaterial implant and determining a first amount of metastasis; (iii) administering the treatment to the subject; (iv) removing the second biomaterial implant and determining a second amount of metastasis; (v) wherein the treatment is effective to reduce metastasis if the second amount of metastasis is lower than the first amount of metastasis. In some examples, the first amount of metastasis and the second amount of metastasis are determined by one or more of inverse-scattering optical coherence tomography (ISOCT), fluorescence activated cell sorting (FACS), high frequency ultrasound, ultrasound, positron emission tomography (PET) scan, magnetic resonance imaging (MRI), photoacoustic imaging, or fluorescence imaging.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1: Physical characteristics and dynamic immune cell response following implantation of micro-porous PCL scaffolds into the dorsal subcutaneous space of a BALB/c mouse. Photomicrograph (A) and scanning electron micrograph (B) of a microporous PCL scaffold. SEM image shows the interconnected porous structure. (C) CD45⁺ leukocyte numbers and (D) Dynamics of CD11b⁺F4/80⁺, CD11c⁺ F4/80⁻, CD11b⁺Gr-1hiLy6C⁻, Ly6C⁺ F4/80⁻, CD4⁺, CD8⁺, CD19⁺, and CD49b⁺ immune cell populations expressed as a percentage of live CD45⁺ leukocytes at day 3, 7, 14, 30, and 60 post PCL scaffold implantation (N≥6 for each time point examined, *p<0.05 compared to day 3 as determined by the Tukey-HSD test post ANOVA). Error bars denote s.e.m.

FIG. 2: Micro-porous scaffolds implanted for 30 days prior to tumor inoculation recruit metastatic cells. Number of (A) total cells and (B) tumor cells (tdTomato+ cells) isolated from micro-porous PLG and PCL scaffolds at day 15 post tumor inoculation analyzed via flow cytometry (N=10, *p<0.01 as determined by t-test for analysis of total cell numbers and Wilcoxon rank-sum test for tumor cell numbers). Fluorescence image of a PCL scaffold section shows the presence of a tumor cell (indicated by white arrow) as identified using tdTomato (C) and DAPI (D) fluorescence and their co-localization (E). Scale bar indicates 20 μm. Error bars denote s.e.m.

FIG. 3: Tumor progression influences dynamics of leukocyte populations at the PCL scaffold. Percentage of (A) CD11b⁺F4/80⁺ (B) CD11c⁺ F4/80⁻ (C) Gr-1^{hi}CD11b⁺ Ly6C⁻ (D) Ly6C⁺ F4/80⁻ innate immune cell populations and percentage of (E) CD4⁺ (F) CD8⁺ (G) CD19⁺ and (H) CD49b⁺ adaptive immune cell populations in the total population of live CD45⁺ leukocytes at day 0, 3, 7, 14, and 21 post tumor inoculation (N≥8 for each time point examined, *p<0.05 compared to day 0 and #p<0.05 compared to day 3 as determined by Tukey-HSD test post ANOVA). Error bars denote s.e.m.

FIG. 4: Micro-porous PCL scaffolds enable early detection of metastatic cells in a chronic model of scaffold implantation. (A) Number of mice with detectable tumor cells analyzed by flow cytometry in the lung, liver, and brain in a group of 5 mice at day 5 post tumor inoculation (N=5 for lung, brain, and liver; N=10 for PCL scaffolds, *p<0.05 as determined using the Fisher's exact test). (B) Percentage of tdTomato+ tumor cells isolated from the PCL scaffold at day 5 post tumor inoculation analyzed via flow cytometry. (C) Average D value for PCL scaffolds isolated from tumor free and tumor bearing mice. Scaffolds from tumor bearing mice were isolated at day 5 post tumor inoculation. (N=14 scaffolds for tumor free and N=16 scaffolds for tumor bearing mice, *p<0.05 as determined using the Wilcoxon rank-sum test). Representative three dimensional maps of D generated via ISOCT analysis of PCL scaffolds in tumor free (D) and tumor bearing mice (E). Scale bars indicate 200 μm. Error bars denote s.e.m.

FIG. 5: Recruitment of 4T1 tumor cells to the PCL scaffold site reduces tumor burden in metastatic sites such as the liver and brain in a chronic model of scaffold implantation in BALB/c mice. Normalized average tumor burden in the (A) liver, (B) brain, and the (C) lung for the scaffold and mock surgery groups. The average burden in the mock group was set to 1 (N≥6 for each group, * p<0.05 compared to mock surgery as determined by the Wilcoxon rank-sum test). Tumor burden in the lung was identical in both groups. Error bars denote s.e.m.

FIG. 6: Micro-porous PCL scaffolds improve survival in a post-surgical model of breast cancer metastasis. (A) Schematic of experimental design to examine the influence of scaffold implant on survival (B) Average resected tumor weights for mock and scaffold group were identical, p=0.93, t-test) (C) Kaplan-Meier survival curve for mice undergoing mock surgery versus mice receiving a scaffold implant (N=7 for each group, *p<0.05 as determined using the Log rank test). Error bars denote s.e.m.

FIG. 7: Micro-porous PCL scaffolds reduce burden of CD11b+Gr-1hiLy6C− cells in the (A) primary tumor and the (B) spleen in BALB/c mice. The percentage of CD11b+Gr-1hiLy6C− cells in the CD45+ leukocyte population was examined at day 10 post tumor inoculation via flow cytometry and is reported as normalized burden. (N=7 for mock surgery; N=8 for scaffold implant, *p<0.05 as determined using t-test). Error bars denote s.e.m.

FIG. 8: Micro-porous PCL scaffolds persist and maintain a space for extended times in vivo. (A) Representative photomicrographs of micro-porous PLG and PCL scaffolds retrieved from tumor free BALB/c mice at day 98 post scaffold implantation. Average scaffold area at day 0 versus day 98 for PLG and PCL scaffolds when tested in a BALB/c (B) and NSG (C) mouse model. N=4; *p<0.0001 compared to day 0 for PLG scaffolds in BALB/c and NSG mouse; p=0.22 compared to day 0 for PCL scaffolds in BALB/c mouse and p=0.7 compared to day 0 for PCL scaffolds in NSG mouse as determined by t-test. Scaffold area was calculated using dimensions obtained from images of scaffolds taken at day 0 and day 98 post implantation using Image J software (http://imagej.nih.gov/ij/). Error bars denote s.e.m.

FIG. 9: Host response following implantation of micro-porous PCL scaffolds in the dorsal subcutaneous space of an NSG mouse in vivo. (A) CD45+ leukocyte numbers and (B) Dynamics of CD11b⁺F4/80⁺, CD11c⁺F4/80⁻, CD11b⁺Gr-1^{hi}Ly6C⁻, and Ly6C⁺F4/80⁻ populations expressed as a percentage of live CD45⁺ leukocytes at day 30 and day 60 post PCL scaffold implantation (N≥8 for each time point examined, *p<0.05 compared to day 30 as determined by t-test). The relative distribution of immune cell populations was nearly identical between day 30 and day 60 post scaffold implantation. Error bars denote s.e.m.

FIG. 10: Dynamics of immune cell populations in the spleen of BALB/c mice with a PCL scaffold implant at day 0, 5, 10, and 15 post tumor inoculation. Percentage of (A) CD11b⁺F4/80⁺ (B) CD11c⁺ F4/80⁻ (C) CD11b⁺Gr-1^{hi}Ly6C⁻ (D) Ly6C⁺ F4/80⁻ innate immune cell populations and percentage of (E) CD4⁺ (F) CD8⁺ (G) CD19⁺ and (H) CD49b⁺ adaptive immune cell populations in the total population of live CD45⁺ leukocytes. (N≥5 for each time point examined, *p<0.05 compared to day 0 and #p<0.05 compared to day 5 as determined by Tukey-HSD test post ANOVA). Error bars denote s.e.m.

FIG. 11: Micro-porous PCL scaffolds enable recruitment of human MDA-MD-231BR cells in a chronic model of scaffold implantation. (A) Total cell infiltration and (B) Tumor cell infiltration in PLG and PCL micro-porous scaffolds. Scaffolds were retrieved at day 15 post tumor inoculation, which was performed 1 month post scaffold implantation (N=10 for each group, *p<0.05 as determined by t-test for analysis of total cell numbers and Wilcoxon rank-sum test for tumor cell numbers). Error bars denote s.e.m.

### DETAILED DESCRIPTION

Provided are techniques for early detection of metastatic cells using an implanted biomaterial scaffold configured to capture such cells. The scaffolds described here are, unlike prior proposals, characterized by greater stability, which, in at least some examples, results in stability sufficient to provide clinically significant capture time frames. The scaffolds may be formed of slow degrading structures or matrices, to thereby allow for metastatic cell collection over months instead of days. This creates conditions for capturing a greater number of cells, for greater cell aggregation at the point of capture, and for better in vivo imaging, thereby allowing more accurate disease identification, diagnoses, and treatment.

In some embodiments, the scaffold is porous and/or permeable. In some embodiments, the polymeric matrix in the scaffold acts as a substrate permissible for metastasis, colonization, cell growth, etc. In some embodiments, the scaffold provides an environment for attachment, incorporation, adhesion, encapsulation, etc. of agents (e.g., DNA, protein, cells, etc.) that create a metastatic capture site within the scaffold. In some embodiments, agents are released (e.g., controlled or sustained release) to attract circulating tumor cells, metastatic cells, or pre-metastatic cells. With regard to agents (e.g., therapeutic agents) and sustained release, for long term therapy (e.g., days, weeks or months) and/or to maintain the highest possible drug concentration at a particular location in the body, the present disclosure in certain embodiments provides a sustained release depot formulation with the following non-limiting characteristics: (1) the process used to prepare the matrix does not chemically or physically damage the agent; (2) the matrix maintains the stability of the agent against denaturation or other metabolic conversion by protection within the matrix until release, which is important for very long sustained release; (3) the entrapped agent is released from the hydrogel composition at a substantially uniform rate, following a kinetic profile, and furthermore, a particular agent can be prepared with two or more kinetic profiles, for example, to provide in certain embodiments, a loading dose and then a sustained release dose; (4) the desired release profile can be selected by varying the components and the process by which the matrix is prepared; and (5) the matrix is nontoxic and degradable. PEG scaffolds as disclosed herein are also contemplated to function as a scaffold that achieves sustained release of a therapeutically active agent. Accordingly, in some embodiments an agent is configured for specific release rates. In further embodiments, multiple different agents are configured for different release rates. For example, a first agent may release over a period of hours while a second agent releases over a longer period of time (e.g., days, weeks, months, etc.). In some embodiments, and as described above, the scaffold or a portion thereof is configured for sustained release of agents. In some embodiments, the sustained release provides release of biologically active amounts of the agent over a period of at least 30 days (e.g., 40 days, 50 days, 60 days, 70 days, 80 days, 90 days, 100 days, 180 days, etc.). In some embodiments, the scaffold or a portion thereof is configured to be sufficiently porous to permit metastasis of cells into the pores. The size of the pores may be selected for particular cell types of interest and/or for the amount of ingrowth desired and are, for example without limitation, at least about 20 μm, 30 μm, 40 μm, 50 μm, 100 μm, 200 μm, 500 μm, 700 μm, or 1000 μm. In some embodiments, the PEG gel is not porous but is instead characterized by a mesh size that is, e.g., 10 nanometers (nm), 15 nm, 20 nm, 25 nm, 30 nm, 40 nm, or 50 nm.

The effectiveness of the longer lifetime scaffolds herein, especially for use as targeted treatment sites, relates to Paget's “seed and soil” paradigm which proposes that, prior to colonization by metastatic cells, supportive cells (e.g., fibroblasts, immune cells, endothelial cells), soluble factors, and extracellular matrix (ECM) components establish a microenvironment conducive to tumor cell homing and colonization. The importance of this paradigm is that metastasis to specific organs is not random, but rather is influenced by the properties of the local environment. The use of longer lifetime scaffolds, in vivo, provides sufficient time for these support cells to establish the microenvironment, at the scaffold, to which metastatic cells are attracted. The initial translation of these principals led to the development and implementation of micro-porous poly(lactide-co-glycolide) (PLG) biomaterial scaffolds, as described in U.S. application Ser. No. 13/838,800, which is hereby incorporated by reference in its entirety. There, the recruitment of metastatic breast cancer cells through the local immune response in vivo was demonstrated. However, PLG scaffolds were degradable over time scales considered too short for clinical translation.

With the present scaffolds and techniques, life times that are well within the timeframe of clinical significance are demonstrated. For example, as discussed herein, stability lifetimes of greater than 90 days are contemplated, with percent degradation profiles of less than about 50%, 45%, 40%, 35%, 30%, 25%, 20%, 15%, 10%, 5%, and 1% respectively, where the percent degradation refers to the scaffolds' ability to maintain its structure for sufficient cell capture as a comparison of its maximum capture ability. Such ability is measured, for example, as the change in porous scaffold volume over time, the change in scaffold mass over time, and/or the change in scaffold polymer molecular weight over time. These long life times mean that scaffolds can now be applied in patient-friendly conditions that allow subjects to wear the scaffold under normal daily living conditions, inside and outside the clinical environment.

Further still, with the present techniques scaffolds are provided that remain functionalized long enough to provide targeted treatment sites in vivo, that is, locations where metastatic cells are not merely just detected, but over time target, to provide a specific location for cell resection and possible removal of all metastatic cells.

The ability of the present scaffolds to remain functionalized over greater periods of time has, in some examples, provided for formation of a sustained or controllable release scaffold. These scaffolds, for example, may comprise protein responsive materials that are non-degradable when implanted and recruiting metastatic cells. When exposed to activating proteins (e.g., an enzyme), however, these scaffolds degrade to then release the captured metastatic cells. In some examples, such a property is contemplated for use in vitro to facilitate the recovery of the captured cells. For example and without limitation, in some examples the scaffold is an alginate scaffold and the activating protein is alginate lyase.

In some examples, the present techniques provide a scaffold formed partially or exclusively of a micro-porous poly(ε-caprolactone) (PCL), forming a PCL scaffold. Such PCL scaffolds have a greater stability than the micro-porous poly(lactide-co-glycolide) (PLG) biomaterial scaffolds, as we show. The PCL scaffolds, in fact, provide such an unexpectedly greater amount of stability that, for the first time, we are able to investigate the dynamic immune response of a subject, as well as other cellular events associated recruitment of metastatic cells. In some of these examples, we have specifically observed cellular events for breast cancer cells.

In some examples, the present invention provides biomaterial PCL and/or PEG and/or alginate scaffolds and implants derived therefrom to provide a target used to recruit and detect metastatic cells. In some examples, that target is used in vitro, in vivo, in situ, etc. In some examples, the present invention provides a biomaterial PCL and/or PEG and/or alginate scaffold used to capture metastatic cells and allow those cells to colonize at a metastatic site over time. In further examples, at least 1, at least 2, at least 3, at least 4, at least 5, at least 6, at least 7, at least 8, at least 9, or at least 10 scaffolds are implanted in a subject.

In some examples, the present techniques provide a controlled release scaffold formed partially or exclusively of hydrogel, e.g., a poly(ethylene glycol) (PEG) hydrogel to form a PEG scaffold. Any PEG is contemplated for use in the compositions and methods of the disclosure. In general, the PEG has an average molecular weight of at least about 5,000 daltons. In further embodiments, the PEG has an average molecular weight of at least 10,000 daltons, 15,000 daltons, and is preferably between 5,000 and 20,000 daltons, or between 15,000 and 20,000 daltons. Also preferred is PEG having an average molecular weight of 5,000, of 6,000, of 7,000, of 8,000, of 9,000, of 10,000, of 11,000, of 12,000 of 13,000, of 14,000, or of 25,000 daltons. In further embodiments, the PEG is a four-arm PEG. In preferred embodiments, each arm of the four-arm PEG is terminated in an acrylate, a vinyl sulfone, or a maleimide. It is contemplated that use of vinyl sulfone or maleimide in the PEG scaffold renders the scaffold resistant to degradation. It is further contemplated that use of acrylate in the PEG scaffold renders the scaffold susceptible to degradation.

In some embodiments, one or more agents are associated with a scaffold to establish a hospitable environment for metastasis and/or to provide a therapeutic benefit to a subject. Agents may be associated with the scaffold by covalent or non-covalent interactions, adhesion, encapsulation, etc. In some embodiments, a scaffold comprises one or more agents adhered to, adsorbed on, encapsulated within, and/or contained throughout the scaffold. The present invention is not limited by the nature of the agents. Such agents include, but are not limited to, proteins, nucleic acid molecules, small molecule drugs, lipids, carbohydrates, cells, cell components, and the like. In various embodiments, the agent is a therapeutic agent. In some embodiments, two or more (e.g., 3, 4, 5, 6, 7, 8, 9, 10 . . . 20 . . . 30 . . . 40 . . . , 50, amounts therein, or more) different agents are included on or within the scaffold. In some embodiments, agents associated with a scaffold include metastatic markers, such as: CD133 (which generally defines all progenitors), VEGFR-1 (hematopoietic progenitor cells (HPCs)), VEGFR-2 (endothelial progenitor cells (EPCs)), CD11b and GR1 (myeloid-derived suppressor cells), F4/80 and CD11b (macrophages), and CD11b+CD115+Ly6c+(inflammatory monocytes).

In further embodiments, it is contemplated that a scaffold of the disclosure recruits more and/or different cells relative to a scaffold that comprises, e.g., PLG. For example, in some embodiments a scaffold of the disclosure recruits more tumor cells than a scaffold that comprises, e.g., PLG. In various embodiments, a scaffold of the disclosure recruits and/or captures about 5, 10, 20, 50, 100, 200, 500, 1000 or more cells relative to a scaffold that comprises, e.g., PLG. In some embodiments, the types of cells that associate with a scaffold of the disclosure are different from a scaffold that comprises, e.g., PLG. For example and without limitation, a higher percentage of CD49b cells are found in association with a PCL scaffold relative to a PLG scaffold; further, there are about equal quantities of F4/80 and CD11c cells in association with a PLG scaffold, whereas there are three times as many CD11c cells as F4/80 cells in association with a PCL scaffold.

The disclosure also contemplates a scaffold which comprises a therapeutic agent. “Therapeutic agent” as used herein means any compound useful for therapeutic purposes. The term as used herein is understood to mean any compound that is administered to a subject for the treatment of a condition.

The present disclosure is applicable to any therapeutic agent for which delivery is desired. Non-limiting examples of such agents as well as hydrophobic drugs are found in U.S. Pat. No. 7,611,728, which is incorporated by reference herein in its entirety. Additional therapeutic agents contemplated for use are found in PCT/US2010/55018, which is incorporated by reference herein in its entirety.

Scaffolds and methods disclosed herein, in various embodiments, are provided wherein the scaffold comprises a multiplicity of therapeutic agents. In some aspects, compositions and methods are provided wherein the multiplicity of therapeutic agents are specifically associated with one scaffold. In other aspects, the multiplicity of therapeutic agents are associated with more than one scaffold.

Therapeutic agents include but are not limited to hydrophilic and hydrophobic compounds.

Protein therapeutic agents include, without limitation peptides, enzymes, structural proteins, receptors and other cellular or circulating proteins as well as fragments and derivatives thereof, the aberrant expression of which gives rise to one or more disorders. Therapeutic agents also include, as one specific embodiment, chemotherapeutic agents. Therapeutic agents also include, in various embodiments, a radioactive material. The term “peptide” as used herein typically refers to short polypeptides/proteins.

In various aspects, protein therapeutic agents include cytokines, chemokines, and/or hematopoietic factors. Cytokines and chemokines are delivered, in various embodiments, to enhance or limit recruitment of cells to the scaffold. Examples of such agents include without limitation IL-1 alpha, IL-1 beta, IL-2, IL-3, IL-4, IL-5, IL-6, IL-11, chemokine (C-C motif) ligand 22 (CCL22), chemokine (C-C motif) ligand 21 (CCL21), chemokine (C-C motif) ligand 2 (CCL2), colony stimulating factor-1 (CSF-1), M-CSF, SCF, granulocyte-macrophage colony-stimulating factor (GM-CSF), granulocyte colony stimulating factor (G-CSF), monocyte chemoattractant protein-1 (MCP-1), interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, erythropoietin (EPO), thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof. Examples of biologic agents include, but are not limited to, immuno-modulating proteins such as cytokines, monoclonal antibodies against tumor antigens, tumor suppressor genes, and cancer vaccines. Examples of interleukins that may be used in conjunction with the compositions and methods of the present invention include, but are not limited to, interleukin 2 (IL-2), and interleukin 4 (IL-4), interleukin 12 (IL-12). Other immuno-modulating agents other than cytokines include, but are not limited to bacillus Calmette-Guerin, levamisole, and octreotide.

As contemplated by the present disclosure, in some aspects therapeutic agents include small molecules. The term “small molecule,” as used herein, refers to a chemical compound, for instance a peptidometic that may optionally be derivatized, or any other low molecular weight organic compound, either natural or synthetic. Such small molecules may be a therapeutically deliverable substance or may be further derivatized to facilitate delivery.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, or about 1000 Daltons.

In various embodiments, therapeutic agents contemplated for use in the compositions and methods disclosed herein and include, but are not limited to, alkylating agents, antibiotic agents, antimetabolic agents, hormonal agents, and plant-derived agents.

Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (e.g., carboplastin, cisplatin and platinum (IV) (Pt(IV))).

Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin.

Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, imatinib mesylate (or GLEEVEC®), and gemcitabine.

Examples of hormonal agents include, but are not limited to, synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), camptothecin compounds (e.g., 20(S) camptothecin, topotecan, rubitecan, and irinotecan), taxanes (e.g., paclitaxel and docetaxel).

Chemotherapeutic agents contemplated for use include, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinum coordination complexes such as cisplatin, Pt(IV) and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

We now describe an example fabrication, characterization, and implantation of micro-porous scaffolds in accordance with examples herein.

In certain embodiments, the scaffold comprises a polymeric matrix. In some embodiments, the matrix is prepared by a gas foaming/particulate leaching procedure, and includes a wet granulation step prior to gas foaming that allows for a homogeneous mixture of porogen and polymer and for sculpting the scaffold into the desired shape.

Thus, in some aspects, the scaffolds may be formed of a biodegradable polymer, e.g., PCL, that is fabricated by emulsifying and homogenizing a solution of polymer to create microspheres. Other methods of microsphere production are known in the art and are contemplated by the present disclosure. See, e.g., U.S. Patent Application Publication Numbers 2015/0190485 and 2015/0283218, each of which is incorporated herein in its entirety. The microspheres are then collected and mixed with a porogen (e.g., salt particles), and the mixture is then pressed under pressure. The resulting discs are heated, optionally followed by gas foaming. Finally, the salt particles are removed. The fabrication provides a mechanically stable scaffold which does not compress or collapse after in vivo implantation, thus providing proper conditions for cell growth.

In some aspects, the scaffolds are formed of a substantially non-degradable polymer, e.g., PEG. Degradable hydrogels encapsulating gelatin microspheres may be formed based on a previously described Michael-Type addition PEG hydrogel system with modifications [Shepard et al., Biotechnol Bioeng. 109(3): 830-9 (2012)]. Briefly, four-arm poly(ethylene glycol) vinyl sulfone (PEG-VS) (20 kDa) is dissolved in 0.3 M triethanolamine (TEA) pH 8.0 at a concentration of 0.5 mg/μL to yield a final PEG concentration of 10%. The plasmin-degradable trifunctional (3 cysteine groups) peptide crosslinker (Ac-GCYKNRCGYKNRCG) is dissolved in 0.3 M TEA pH 10.0 to maintain reduction of the free thiols at a concentration that maintain a stoichiometrically balanced molar ratio of VS:SH. Prior to gelation, gelatin microspheres are hydrated with 10 μL sterile Millipore or lentivirus solution. Subsequently, the PEG and peptide crosslinking solutions are mixed well and immediately added to the hydrated gelatin microspheres for encapsulation. In some embodiments, and as described above, salt is used as the porogen instead of gelatin microspheres. In this case, the PEG solution is made in a saturated salt solution, so that the porogen does not significantly dissolve.

In some embodiments, UV crosslinking is used instead of peptide crosslinking. Ultraviolet crosslinking is contemplated for use with PEG-maleimide, PEG-VS, and PEG-acrylate.

Following production but prior to use of a PCL scaffold, the scaffold is weighed to ensure that the salt is gone. The integrity of the PCL scaffold is also evaluated by its handling; the scaffold is viewed under a microscope to examine the pore structure. Similarly, following production but prior to use of a PEG scaffold, its integrity is evaluated by handling and viewing the scaffold under a microscope to see the pore structure.

Scaffolds of the present disclosure may comprise any of a large variety of structures including, but not limited to, particles, beads, polymers, surfaces, implants, matrices, etc. Scaffolds may be of any suitable shape, for example, spherical, generally spherical (e.g., all dimensions within 25% of spherical), ellipsoidal, rod-shaped, globular, polyhedral, etc. The scaffold may also be of an irregular or branched shape.

In some embodiments, a scaffold comprises nanoparticles or microparticles (e.g., compressed or otherwise fashioned into a scaffold). In various embodiments, the largest cross-sectional diameters of a particle within a scaffold is less than about 1,000 μm, 500 μm, 200 μm, 100 μm, 50 μm, 20 μm, 10 μm, 5 μm, 2 μm, 1 μm, 500 nm, 400 nm, 300 nm, 200 nm or 100 nm. In some embodiments, a population of particles has an average diameter of: 200-1000 nm, 300-900 nm, 400-800 nm, 500-700 nm, etc. In some embodiments, the overall weights of the particles are less than about 10,000 kDa, less than about 5,000 kDa, or less than about 1,000 kDa, 500 kDa, 400 kDa, 300 kDa, 200 kDa, 100 kDa, 50 kDa, 20 kDa, 10 kDa.

In some embodiments, a scaffold comprises PCL. In further embodiments, a scaffold comprises PEG. In certain embodiments, PCL and/or PEG polymers and/or alginate polymers are terminated by a functional group of chemical moiety (e.g., ester-terminated, acid-terminated, etc.).

In some embodiments, the charge of a matrix material (e.g., positive, negative, neutral) is selected to impart application-specific benefits (e.g., physiological compatibility, beneficial interactions with chemical and/or biological agents, etc.). In certain embodiments scaffolds are capable of being conjugated, either directly or indirectly, to a chemical or biological agent). In some instances, a carrier has multiple binding sites (e.g., 2, 3, 4, 5, 6, 7, 8, 9, 10 . . . 20 . . . 50 . . . 100, 200, 500, 1000, 2000, 5000, 10,000, or more).

In some embodiments, the present disclosure provides methods for detection of a metastatic cancer cell on an implanted scaffold. In general, any method useful for detection of a metastatic cell in a scaffold may be used. In some embodiments, non-invasive methods of metastasis detection are provided. In some embodiments, adapt inverse-scattering optical coherence tomography (ISOCT) is provided for non-invasive scaffold imaging. In certain embodiments, ISOCT enables three-dimensional (3D) imaging of tissue microvasculature and ultrastructure with detail that enables detection of a metastatic cell to, upon, or within scaffolds. In further embodiments, high frequency ultrasound, ultrasound, or photoacoustic imaging are utilized for scaffold imaging.

In some embodiments, and as described herein, compositions and methods of the present disclosure provide a sensor of metastasis in a subject (e.g., a subject suspected of having cancer, a subject with cancer, a subject in remission, a subject not necessarily at elevated risk of cancer or metastasis). In some embodiments, a composition is implanted within a subject and metastasis thereto is monitored to detect metastasis within the subject. In some embodiments, a scaffold is implanted and checked at regular (e.g., daily, semi-daily, weekly, etc.) or periodic intervals (e.g., monthly, yearly, etc.) for evidence of metastasis. In some embodiments, a single scaffold is monitored over time for changes in the metastatic state thereof. In some embodiments, scaffolds are implanted and removed following procedures to detect metastasis. Removal of a scaffold occurs, in various embodiments, after about 1 day, 2 days, 3 days, 4 days, 5 days, 6 days, 7 days, 8 days, 9 days, 10 days, 11 days, 12 days, 13 days, 14 days, 15 days, 3 weeks, 4 weeks, 5 weeks, 6 weeks, 7 weeks, two months, three months, six months, eight months, one year, two years, three years, four years, five years, or more. In further embodiments, removal of a scaffold is followed by a determination of the number of metastatic cells in the scaffold, and then the scaffold is replaced in the subject in the same or a different location from which it was removed.

In some embodiments, removal of a scaffold is followed by retrieval of the captured cells. Such captured cells are, in various embodiments, cancer cells and/or non-cancer cells. Retrieval of the cells is achieved as disclosed herein and typically involves exposure of the scaffold to an activating protein such as an enzyme. Following retrieval, the captured cells may be studied for development of a cancer vaccine. In further embodiments, analysis of the captured cells are indicative of the type of cancer from which the subject is suffering. Elucidation of the type of cancer a subject is suffering from is contemplated to inform the type of therapy (e.g., surgery, targeted chemotherapy, radiotherapy, and/or personalized/precision therapy) the subject should receive.

### Example Implementation

To demonstrate the efficacy of our proposed scaffolds as capable of attracting metastatic cells, an orthotopic xenotransplant model of human breast cancer metastasis in female NSG mice was developed. The metastatic human cell line used for studies conducted during development of embodiments of the present invention was MDA-MB-231-BR (231BR), a spontaneously metastasizing variant of the triple-negative MDA-MB-231 breast cancer line, which has previously undergone selection for its ability to metastasize to the brain. The 231BR cell line was then stably transfected to express luciferase and tdTomato to generate the MDA-MB-231BR-tdTomato-luc2 cell line.

Scaffold Fabrication: For preparation of microporous PCL scaffolds, PCL microspheres were first prepared by emulsifying a 6% (w/w) solution of PCL (Lactel Absorbable Polymers, Birmingham, Ala.; Inherent viscosity=0.65-0.85 dL/g) in dichloromethane in a 10% poly(vinyl alcohol) solution followed by homogenization at 10,000 rpm for ˜1 min. The solution was then stirred for at least 3 h. Microspheres were collected by centrifugation and washed at least 5 times in deionized water, followed by lyophilization for 48 h. To prepare micro-porous PCL scaffolds, PCL microspheres and salt particles (size range 250-425 μm) were mixed in a 1:30 (w/w) ratio and pressed at 1500 psi in a steel die for approximately 45 s. Polymer-salt discs were heated at 60° C. for approximately 5 min on each side, followed by foaming in high pressure CO₂ at 800 psi for approximately 24 hours. Salt particles were removed by immersing discs in water. Scaffolds were stored in −80° C. For experimental studies, scaffolds were sterilized using 70% ethanol, rinsed with sterile water, and dried on a sterile gauze pad.

The microporous PLG scaffolds, used for comparison, were prepared using gas foaming and particulate leaching techniques as described in U.S. application Ser. No. 13/838,800.

Scaffold Characterization: Mechanical testing of scaffolds was performed on a Sintech Instron 20/G (Instron Corp, Norwood Mass.) at a crosshead rate of 1 mm/min with a 200 g load cell. Samples were compressed to 50% strain and the Young's Modulus of the scaffolds was calculated from the linear region of stress/strain as determined by TestWorks 4 (MTS Systems Corp, Eden Prairie, Minn.) software. For examining scaffold micro-architecture, scaffolds were coated with 15 nm gold and imaged using a scanning electron microscope (Hitachi S4800-II cFEG SEM; Hitachi High-Technologies) at an accelerating voltage of 2 kV. Scaffold porosity was calculated using the equation: Porosity=1−(ρ/ρ*); where ρ is the density of the polymer and ρ* is the apparent density of the scaffold (scaffold weight/scaffold volume). Pore volume was calculated using the equation: Pore volume=Scaffold volume×Porosity.

Scaffold Implantation: Microporous scaffolds were implanted in the subcutaneous space of either BALB/c mice or NOD/SCID-IL2Rγ^{-/-} (NSG) mice. For the implantation procedure, mice were anesthetized with an intraperitoneal injection of Ketamine (10 mg/kg) and Xylazine (5 mg/kg). The upper back was shaved and prepped using a betadine swab followed by an ethanol swab (3×). An incision was made in the upper back and a subcutaneous pocket was created on each side, into which the scaffolds were inserted (2 scaffolds per mouse). The skin was closed using wound clips (Reflex 7 mm, Roboz Surgical Instrument Co.) and surgical glue (3M Vetbond Tissue Adhesive).

Orthotopic tumor inoculation was performed one month after scaffold implantation. For tumor inoculation, an incision was made along the right side of the lower half of the dorsal skin. Subsequently, a subcutaneous pocket was created and the right fourth mammary fat pad was exposed. 2×10⁶ 4T1-luc2-tdTomato (Perkin Elmer) or MDA-MB-231BR-tdTomato-luc2 cells in 50 μL sterile phosphate buffer saline (PBS) (Life Technologies) were then injected into the fourth right mammary fat pad of a female BALB/c or an NSG mouse. The skin was then closed with surgical glue (3M Vetbond Tissue Adhesive).

Flow cytometry: Mice were euthanized at indicated times and the retrieved scaffolds and organs were washed in Hank's Balanced Salt Solution (Life Technologies). Samples were minced using micro scissors in Liberase TL or TM (0.38 mg/mL) (Roche) and placed at 37° C. for 20 min. Following this, 0.5M EDTA (Life Technologies) was added and cells were retrieved from tissues or scaffolds by straining through a 70 μm filter in FACS buffer [PBS (Life Technologies) with 0.5% Bovine Serum Albumin (Sigma Aldrich) and 2 mM EDTA]. For analysis of tumor cells (tdTomato+cells), individual scaffold or organ samples were suspended in FACS buffer and analyzed using a BD LSR Fortessa flow cytometer (BD Biosciences). The detection sensitivity for cancer cells via flow cytometry was 0.002% (i.e., 5 cancer cells in 250,000 total cells). For analysis of various leukocyte populations, individual scaffold samples were split equally for analyzing innate and adaptive immune cells. In each set, cells were blocked using anti-CD16/32 (1:50, eBioscience) and stained using LIVE/DEAD® Fixable Blue Dead Cell Stain Kit (1:200, Life Technologies). In the first set, cells were stained with Alexa Fluor® 700 anti-CD45 (1:125, Biolegend), V500 conjugated anti-CD11b (1:100, BD Biosciences), FITC conjugated anti-Ly6C (1:100, Biolegend), PE-Cy7 conjugated anti-F4/80 (1:80, Biolegend), APC conjugated anti-CD11c (1:80, Biolegend), and Pacific Blue™ anti-Ly-6G/Ly-6C (Gr-1) (1:70, Biolegend). In the second set, cells were stained with Alexa Fluor® 700 anti-CD45 (1:125, Biolegend), V500 conjugated anti-CD4 (1:100, Biolegend), Pacific Blue™ anti-CD19 (1:100, Biolegend), FITC conjugated anti-CD8 (1:25, Biolegend), and PE-Cy7 conjugated anti-CD49b (1:30, Biolegend). Samples were then analyzed using a BD LSR Fortessa flow cytometer (BD Biosciences). Single color controls were used to set the gating scheme for analysis of leukocyte populations as described previously.

To examine scaffolding targeting ability, initially and over time, a series of scaffold sectioning and fluorescence imaging was performed.

Scaffolds retrieved from mice were rinsed in PBS and then immediately flash frozen in pre-chilled isopentane. Frozen scaffolds were then embedded in optimal cutting temperature (OCT; Cardinal Health) compound with 30% sucrose and sectioned using a cryostat (Microm HM 525; Microm International) at 14 μm. Scaffold sections were stored at −20° C. until imaging. Cryosections were air-dried at room temperature for 30 min, fixed with 10% neutral buffered formalin, washed with tap water for 5 min, DI water for 10 min (2×) and cover slipped with ProLong Gold antifade aqueous mounting medium containing DAPI (Molecular Probes, Grand Island, N.Y.). DAPI fluorescence was visualized using an excitation wavelength of 358 nm, and fluorescence from tdTomato in the cancer cells was visualized using an excitation wavelength of 532 nm. Images were viewed using an Olympus BX43 microscope and an Olympus DP72 digital camera with CellSens Entry software (Olympus) used for image capture and co-localization.

Various techniques may be used to analyze captured cells, in vivo and ex vivo. These include inverse-scattering optical coherence tomography (ISOCT) imaging and ultrasound imaging, the later being particularly useful in sub-surface imaging of scaffolds, i.e., implanted under the skin. We describe an example of non-invasive scaffold imaging using ISOCT imaging. ISOCT enables three-dimensional (3D) imaging of tissue microvasculature and ultrastructure with detail well below the diffraction-limited limit of resolution (sensitivity to length scales as small as 40 nm). ISOCT and scanning transmission electron microscopy (STEM) were performed ex vivo on scaffolds extracted after colonization and control scaffolds in order to identify ISOCT-detectable endogenous ultrastructural and microvascular markers of the scaffold response to cell migration. Further, experiments are conducted to determine the minimal number of malignant cells that induce a microenvironmental change detectable by ISOCT. ISOCT offers a label-free approach to quantify the statistical mass density correlation function of tissue with subdiffractional sensitivity.

In the ISOCT imaging and analysis herein, a spectral domain OCT system with illumination wavelength from 650-800 nm was used to measure backscattering intensity from each 3-D resolution voxel having dimensions 8×8×4 μm. From the backscatter intensity spectrum, the refractive index correlation function shape factor, D was calculated. For label-free imaging of PCL scaffolds in situ, mice were euthanized at day 5 post tumor inoculation. An incision was made to expose the surface of implanted PCL scaffolds to the scanning OCT laser and two 2×2 mm scans were obtained for each scaffold. To minimize random surface reflections, PBS was added onto the scaffold. To determine mean D value from each measurement, lateral regions of each scan free from reflection and excess adiposity were averaged from 8 to 48 μm below the surface of the scanned sample. To generate representative three-dimensional renderings of a scaffold from tumor free and tumor bearing mice, conventional OCT backscatter intensity was mapped in grayscale with a color map overlay of D values. The D map was processed with a 24×24 μm horizontal filter and a 32 μm vertical filter to smoothen the image.

The influence of scaffold implant on survival was investigated using a post-surgical model of breast cancer metastasis. In this model, the primary tumor was resected 10 days post tumor inoculation. Briefly, the primary tumor area was prepped using a betadine swab followed by an ethanol swab (3×). An incision was made along the right side of the lower half of the dorsal skin exposing the primary tumor. The tumor was then picked up using needle nose-forceps and the skin around the base of the tumor was cut using curved tip scissors. The skin was closed using MONOCRYL® (poliglecaprone 25) suture (Ethicon, Inc.) and surgical glue (3M Vetbond Tissue Adhesive). Animal health was monitored daily after the procedure for activity and responsiveness including posture, mobility, body weight, grooming behavior, and respiratory conditions. Animals were euthanized if found in a moribund condition as an experimental endpoint. Mice that evidenced re-growth of the primary tumor were excluded from the analysis to avoid confounding effects arising from the primary tumor.

Statistical analysis: Data are presented as mean±standard error (s.e.m.). Multiple comparisons were performed using one-way ANOVA. Comparisons post ANOVA was performed using the Tukey-HSD test. For data that did not follow a normal distribution, comparison between two samples was performed using the non-parametric Wilcoxon rank-sum test. For comparing the relative number of mice containing detectable tumor cells in organs with scaffolds, a Fisher's exact test was used to determine the p-value. Statistical analysis was performed using JMP Software (JMP Pro 11). For survival analysis, Kaplan-Meier curve was generated and statistical analysis was performed using a Log-rank test using Sigma Plot (Version 13).

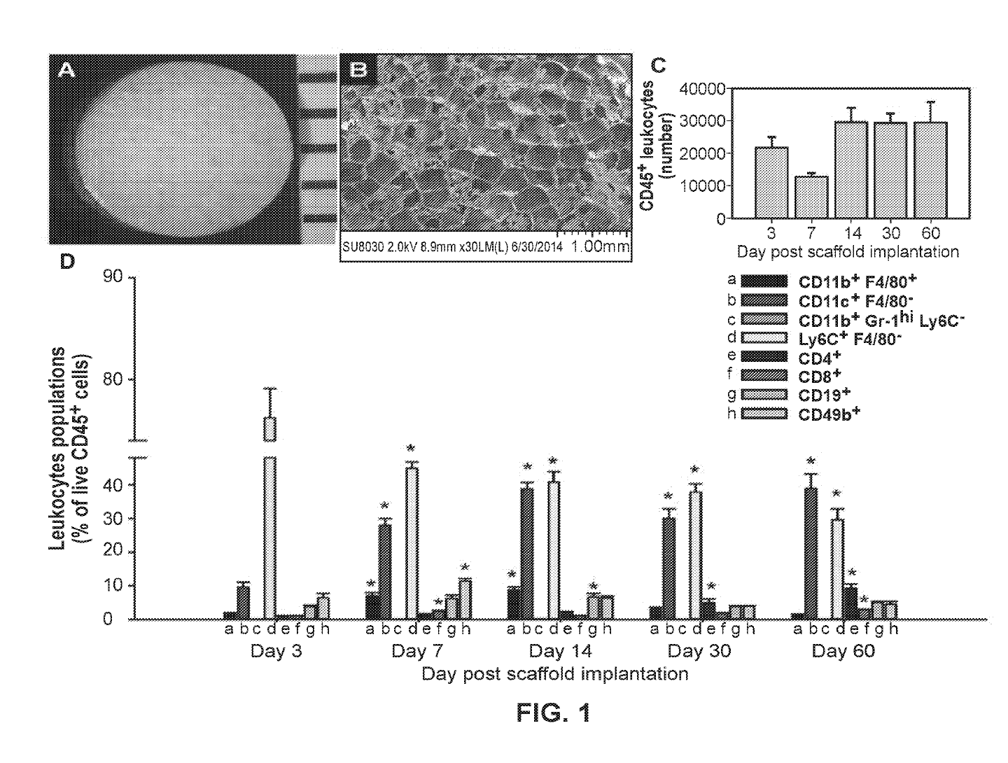

Micro-porous PCL scaffolds (FIG. 1A, 5 mm diameter and 2 mm height) were developed to create microenvironments in vivo and subsequently examine their ability to recruit metastatic tumor cells. The porous interconnected architecture of the scaffold was confirmed using SEM imaging (FIG. 1B). Micro-structural features such as porosity, pore volume, and mechanical properties (i.e., elastic modulus) were similar for PCL and previously reported PLG scaffolds (Table 1).

TABLE 1
Characterization of micro-porous PLG and PCL scaffolds (N = 10).

| | Scaffold Properties | | |
|---|---|---|---|
| | Elastic Modulus | Porosity | Pore Volume |
| Scaffold Type | (KPa) | (%) | (mm³) |
| PLG | 1004 ± 112 | 96.7 ± 0.001 | 38.1 ± 0.04 |
| PCL | 664 ± 97 | 94.7 ± 0.005 | 37.2 ± 0.18 |

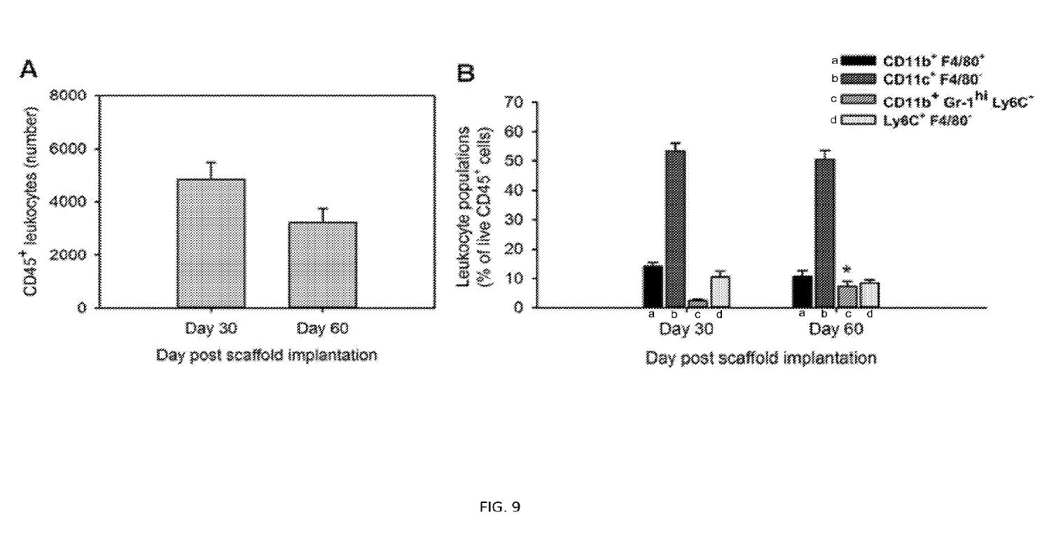

The ability of PCL scaffolds to persist and create a defined space in vivo was investigated by implantation into the subcutaneous dorsal space of BALB/c and NSG mice. The subcutaneous site was selected for its accessibility and amenability to non-invasive imaging. Furthermore, neither 4T1 nor MDA-MB-231BR breast cancer cells typically metastasize to the subcutaneous space, thus the presence of cancer cells in the metastatic site would likely be associated with the presence of the scaffold. PCL scaffolds retrieved after 3 months experienced minimal degradation when compared to day 0 as opposed to PLG scaffolds, which had previously been employed for in vivo recruitment of tumor cells. PLG scaffolds showed significant degradation over this time period as quantified by scaffold area (i.e., 66% in NSG and 77% in BALB/c mouse; FIG. 8).

The dynamic immune response to the biomaterial implant was investigated throughout the acute and chronic phases. Implantation of the PCL scaffold into healthy BALB/c mice resulted in infiltration of CD45⁺ leukocytes by day 3. The number of CD45⁺ leukocytes remained relatively unchanged after day 14 post scaffold implantation (FIG. 1C). However, the relative distribution of leukocyte populations examined, including innate and adaptive immune cells, changed dynamically following scaffold implantation. The percentage of inflammatory monocytes, identified as Ly6C⁺F4/80⁻ cells, decreased after day 3 and remained relatively stable at later time points, whereas the percentage of dendritic cells, identified as CD11c⁺F4/80⁻, increased after day 3 and remained stable at later time points (FIG. 1D). These two cell populations constituted the majority of cells (i.e., ≥65%) observed at the PCL scaffold at later time points. The percentage of macrophages, identified as CD11b⁺ F4/80⁺ cells, significantly increased through day 14 (e.g., 8.8% at day 14 vs. 1.7% at day 3, FIG. 1D, p<0.05) and then returned to levels observed at day 3 (e.g., 1.4% at day 60, FIG. 1D, p=0.99 compared to day 3). In contrast, the levels of myeloid derived suppressor cells (MDSCs) identified by CD11b⁺Gr-1^{hi}Ly6C⁻ staining remained low at all time points examined at 0.15% (FIG. 1D). In the adaptive immune cell population, the percentage of CD4⁺ helper T cells and CD8⁺ cytotoxic T cells significantly increased over time (e.g., 1% at day 3 to 9% at day 60 for CD4⁺ and 1.2% at day 3 to 3% at day 60 for CD8⁺ respectively, FIG. 1D, p<0.05). The percentage of B cells, identified as CD19⁺, and natural killer (NK) cells, identified as CD49b⁺, increased post day 3 and returned to day 3 levels at later time points (i.e., day 30 and 60; FIG. 1D). Importantly, the relative percentages of leukocyte subpopulations were similar between day 30 and day 60 post scaffold implantation in BALB/c mice (FIG. 1D). This trend was also observed in NSG mice (FIG. 9). Based on the stabilization of cell populations after day 30, we utilized day 30 as a time point representing the chronic response to a scaffold implant in all following experiments.

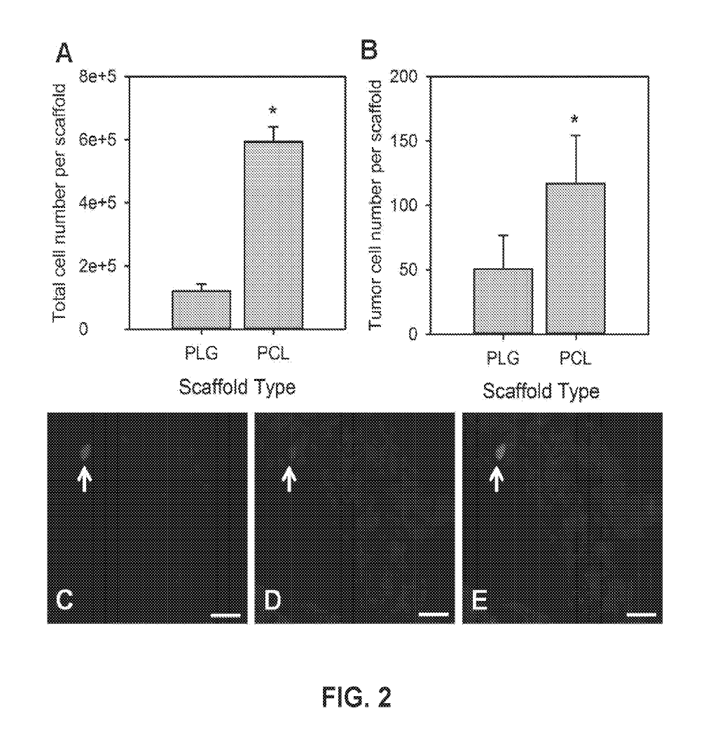

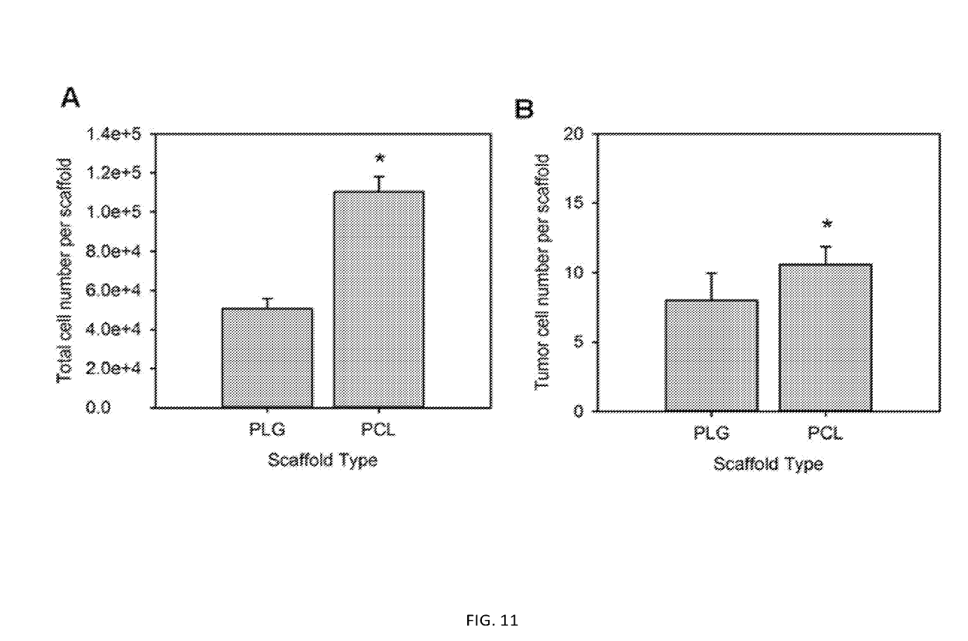

The recruitment of metastatic cells to a chronically implanted microporous scaffold (i.e., a scaffold that had been implanted for 30 days prior to tumor inoculation, a time corresponding to the chronic phase of the immune response) was subsequently examined. Flow cytometry and fluorescence imaging (FIG. 2) performed for scaffolds retrieved at day 15 post tumor inoculation demonstrated the presence of mouse 4T1 tumor cells in the scaffold, indicating that the local microenvironment enabled recruitment of tumor cells. Total cell infiltration was significantly greater within PCL scaffolds compared to PLG scaffolds (i.e., ˜6×10⁵ cells in the PCL scaffold vs. ˜1×10⁵ cells in the PLG scaffold, p<0.0001, FIG. 2A) and a similar trend was observed for tumor cell recruitment (FIG. 2B, p<0.01). Scaffolds were also able to recruit human MDA-MB-231BR cells in NSG mice (FIG. 11), indicating that such a system enabled recruitment of mouse and human breast cancer cells in the context of both immune competent and immune compromised mouse models, respectively.

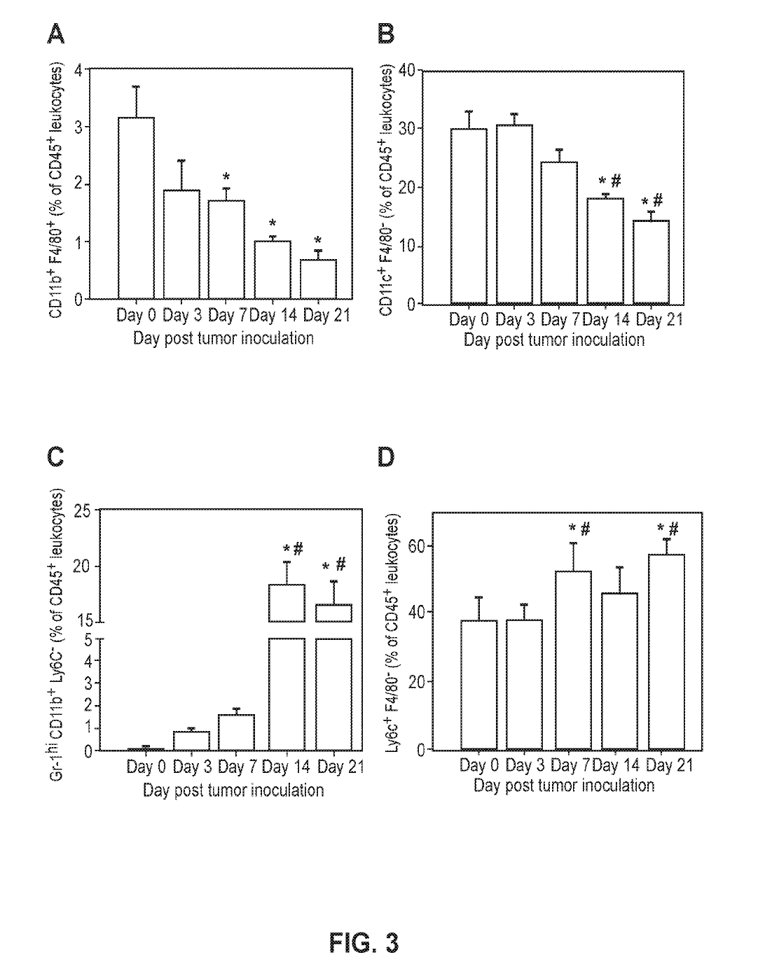

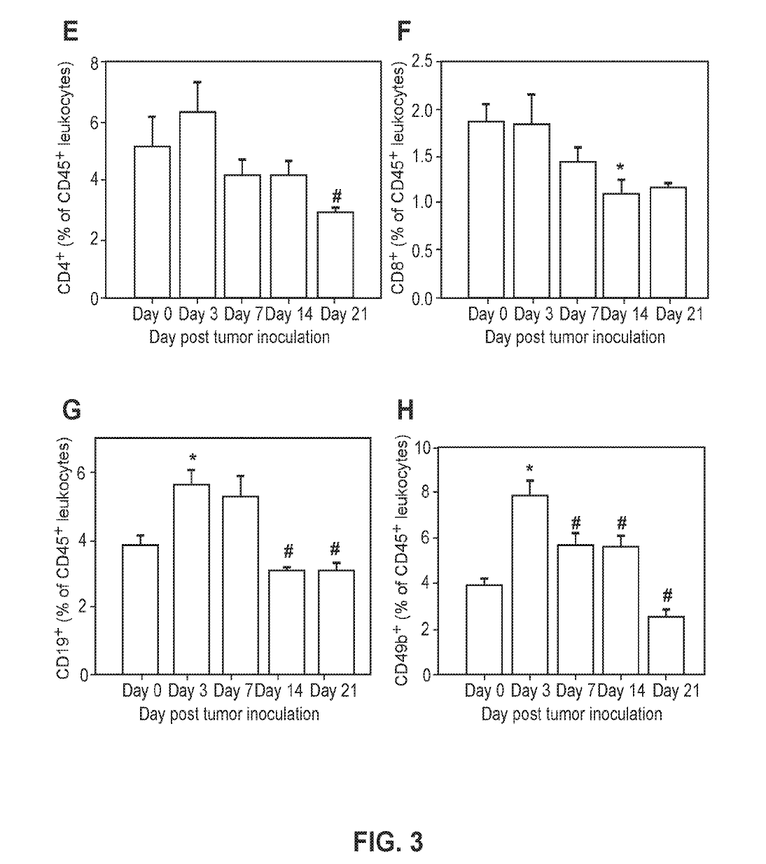

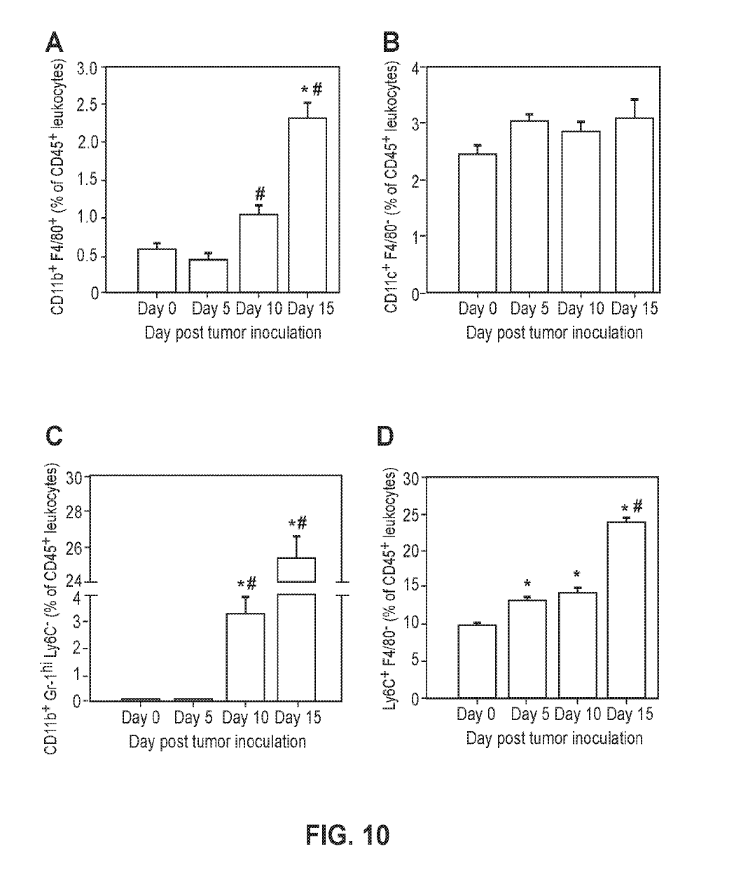

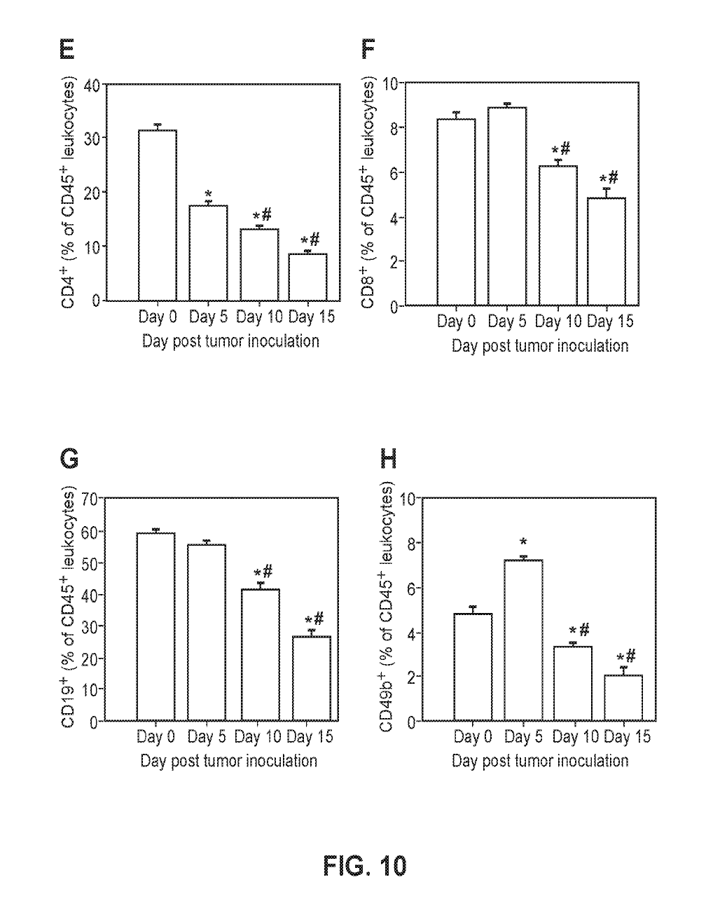

Following tumor inoculation, the dynamics of immune cell populations at the PCL scaffold was subsequently characterized, as tumor cells are known to influence the recruitment of immune cells from the bone marrow. Flow cytometric analysis indicated an increase in Ly6C⁺F4/80⁻ and CD11b⁺Gr-1^{hi}Ly6C⁻ cells at the PCL scaffold site (FIGS. 3C and 3D, p<0.0005). For example, the numbers of CD11b⁺Gr-1^{hi}Ly6C⁻ cells increased from 0.1% at day 0 to 17% at day 21 post tumor inoculation (p<0.05), an increase of two orders of magnitude relative to their numbers at the PCL scaffold site in tumor-free BALB/c mice (FIG. 1D, FIG. 3C). Both cell types have been implicated in the pre-metastatic niche. In contrast, the percentages of CD11b⁺F4/80⁺ macrophages, CD11c⁺F4/80⁻ dendritic cells, and CD8⁺ cytotoxic T cells decreased at the PCL scaffold site (FIG. 3A, 3C, 3F, e.g., 30% at day 0 vs. 14% at day 21 for dendritic cells, p<0.05). The percentage of CD19⁺ B cells, CD49b⁺ NK cells, and CD4⁺ helper T cells increased at day 3 and then decreased at later time points (FIG. 3G, 3H, 3E). Specifically, NK cells increased from 4% at day 0 to 8% at day 3, followed by a decrease to 2.5% at day 21 post tumor inoculation (FIG. 3H, p<0.05). Interestingly, the immune cell dynamics at the PCL scaffold site reflected the dynamics observed in the spleen post tumor inoculation (FIG. 3 vs. FIG. 10). In summary, the changing immune microenvironment at the PCL scaffold site post tumor inoculation correlated with recruitment of 4T1 tumor cells, and is consistent with prior literature reports on the role of the immune cells in the pre-metastatic niche.

One of the benefits of the disclosed techniques is that the scaffolds may be used for early detection of metastatic cells at the PCL scaffold, thereby allowing the scaffold to be used in early stage identification of metastasis. Further, the stability of the PCL and PEG scaffolds mean that they can remain functionalized from these early stages to clinically significant timeframes where metastasis is believed to occur.

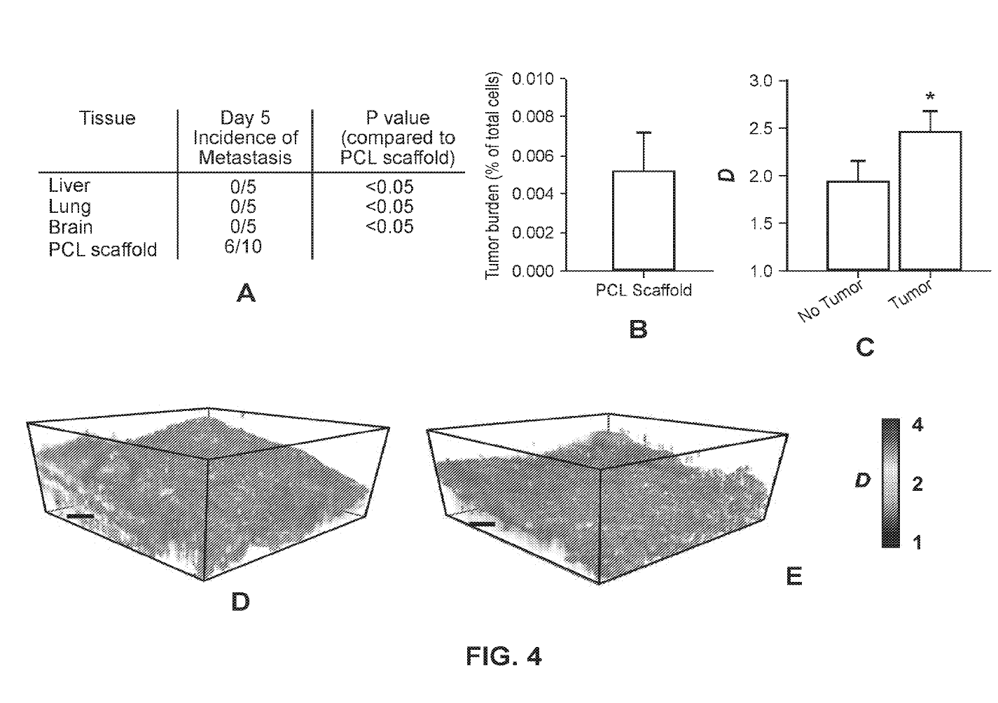

The ability to detect the presence of metastatic disease at an early stage was examined through evaluation of the percentage of tumor cells in the PCL scaffold relative to the cancer cells detected in typical metastatic sites such as the lung, liver, and brain, at day 5 post tumor inoculation. Flow cytometry analysis revealed that the PCL scaffolds had a detectable percentage of tumor cells (i.e., 0.005±0.002%) compared to the lung, liver, and the brain, none of which had detectable tumor cells (FIGS. 4A and 4B; N=5 for lung, liver, and brain, N=10 for PCL scaffolds, p<0.05, Fisher's exact test). The greater density of tumor cells observed at the PCL scaffold site compared to other organ sites supports the use of this tool for detecting metastatic disease at a nascent stage.

The feasibility of using a label-free imaging technique was subsequently investigated. inverse spectroscopic optical coherence tomography (ISOCT), for the early detection of metastatic disease in a chronic model of scaffold implantation. Prior studies of early carcinogenesis with ISOCT and a similar spectroscopic technique, low-coherence enhanced backscattering spectroscopy (LEBS), have revealed that D measured from tissue increases with cancer progression. Thus, similar ultra-structural tissue modifications occurring in the pre-metastatic niche are likely to have a analogous effect on D. D has previously been reported to reflect mass-density distribution features at length scales of 35-350 nm. In addition, D values from tissue have been demonstrated as a robust biomarker of early-stage carcinogenesis. Consistent with these observations and data obtained via flow cytometric analysis (FIGS. 4A and 4B), a significant increase was observed in average D values obtained from ISOCT measurements at the PCL scaffold site in tumor bearing mice (N=7) compared to tumor free mice (N=8; p<0.05, FIG. 4C), confirming ultra-structural alterations to the scaffold and further indicative of the presence of tumor cells. The color map overlay of D values (FIGS. 4D and 4E) demonstrated the distribution throughout the scaffold. These results suggest that ISOCT could be employed for early detection of metastatic disease at the PCL scaffold.

The present PCL scaffolds resulted in implantation that reduced tumor burden and improved disease-specific survival in comparison to PLG scaffolds.

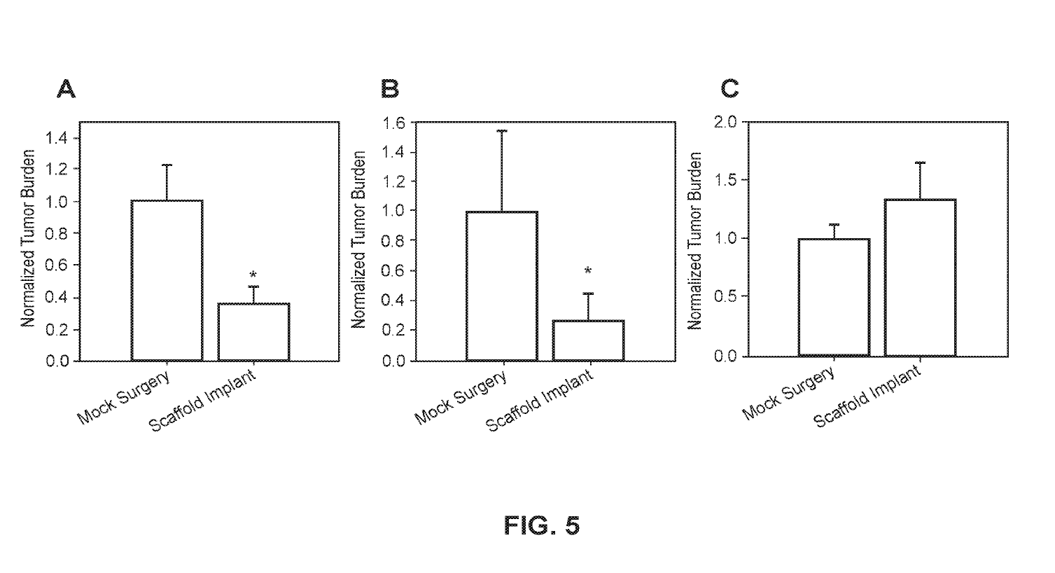

Whether the recruitment of the metastatic cells to the chronically implanted PCL scaffolds may reduce the tumor burden at typical metastatic sites, such as the liver, brain, and the lung at day 15 post tumor inoculation was next investigated. Flow cytometry analysis indicated that the percentage of tumor cells in the liver and the brain was reduced in mice receiving a PCL scaffold versus mice undergoing a mock surgery. As stated, the tumor burden was reduced by 64% for the liver (FIG. 5A, N=15, p<0.05) and 75% for the brain (FIG. 5B, N=8 for mock surgery, N=6 for scaffold implant, p<0.05 as determined using the Wilcoxon rank-sum test in both cases). However, in this immunocompetent mouse model, a reduction in the tumor burden in the lung was not observed (FIG. 5C, N=11, p=0.7) distinct from our previous observations in an immune compromised NSG mouse inoculated with human MDA-MB-231BR cells.

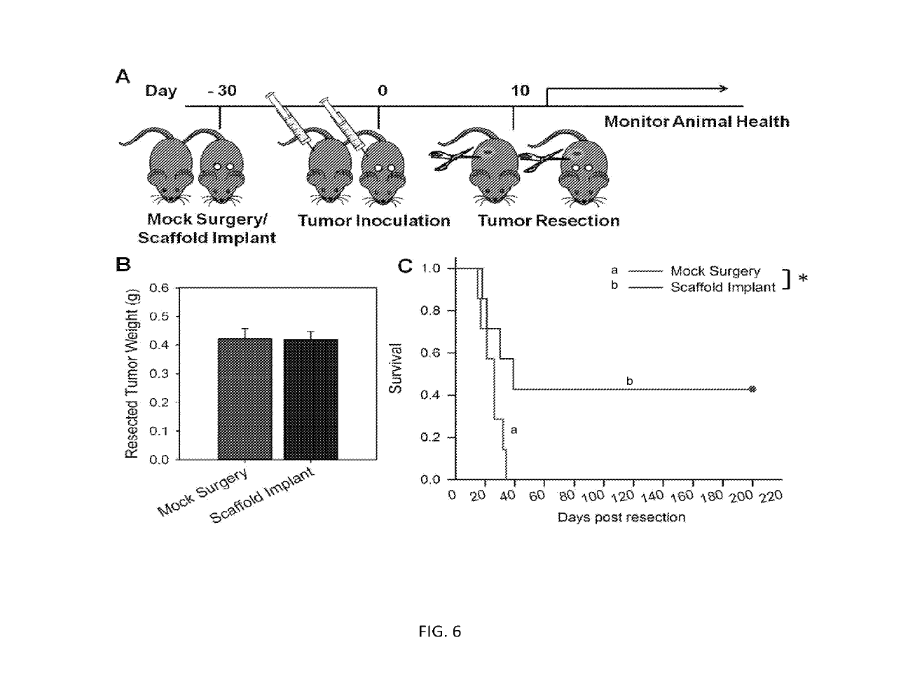

A post-surgical model of breast cancer metastasis was then applied to investigate the potential for PCL scaffold implants to influence survival. In this model, the primary tumor was resected at day 10 post tumor inoculation (FIG. 6A), which corresponded to a time after which cancer cells were detectable in the scaffold by label-free imaging (i.e., day 5, FIG. 4). The resected tumor weights were comparable for both groups, with tumors from the mock surgery group weighing 0.423±0.035 g versus tumors from scaffold implanted mice weighing 0.419±0.029 g (p=0.93, t-test, FIG. 6B). Kaplan-Meier survival analysis demonstrated a significant improvement in survival in mice receiving a PCL scaffold implant compared to mice receiving a mock surgery (FIG. 6C, N=7 per group, p<0.05, Log-rank test). The sacrifice end-points utilized for mice in both groups are described in Table 2.

TABLE 2
Sacrifice end points observed in the post-surgical model in the mock
and scaffold groups.

| Reason for Sacrifice | Mock Group | Scaffold Group |
|---|---|---|
| Hunched posture, bad grooming, | 2/7 | 1/7 |
| and lethargy | | |
| Labored breathing | 3/7 | |
| Hind limb paralysis^{#} | 1/7 | 1/7 |
| Fore limb paralysis^{#} | | 1/7 |
| Cause unknown - mice found | 1/7 | 1/7 |
| dead in cage | | |
| Experiment terminated - mice | — | 3/7 |
| were healthy, no signs of disease | | |
| observed | | |

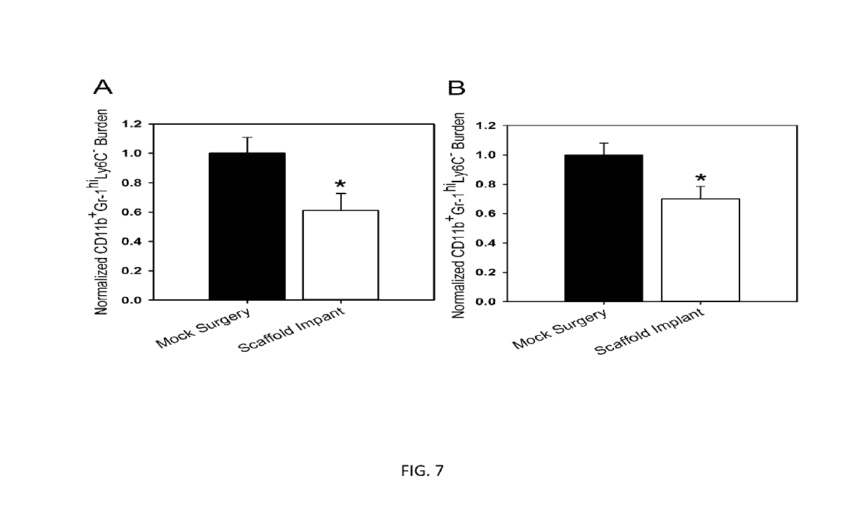

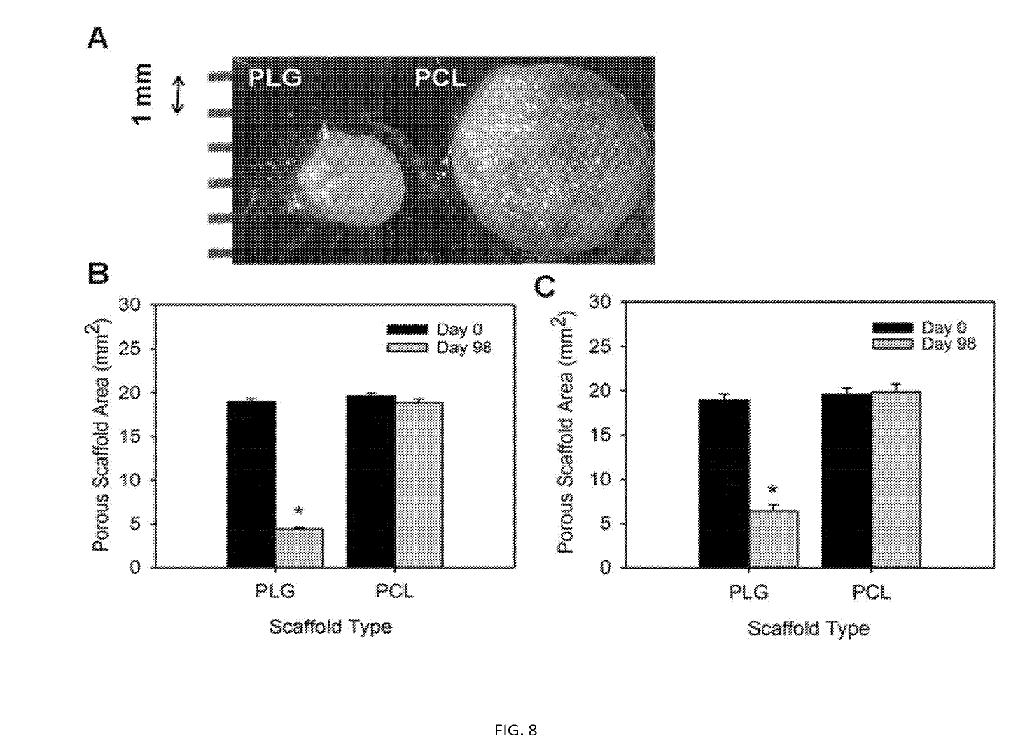

Given the greatest increase in the abundance of CD11b⁺Gr-1^{hi}Ly6C⁻ cells (2 orders of magnitude change) at the PCL scaffold site post tumor inoculation, we hypothesized that the increased survival with scaffold implantation may reflect a differential distribution of CD11b⁺Gr-1^{hi}Ly6C⁻ cells at the primary tumor (local) and the spleen (systemic). Flow cytometric analysis indicated that the abundance of CD11b⁺Gr-1^{hi}Ly6C⁻ cells was reduced in mice receiving a scaffold implant versus mice receiving a mock surgery examined at day 10 post tumor inoculation. The burden of CD11b⁺Gr-1^{hi}Ly6C⁻ cells was reduced by 39% in the primary tumor (FIG. 7A) and 30% in the spleen (FIG. 7B, N≥7, p<0.05 as determined by t-test in both cases). This result suggests that, in part, presence of the scaffold contributes to a reduction in the abundance of key niche cells locally (i.e., primary tumor site) and systemically (i.e., spleen) that support metastasis. Taken together, these results highlight the potential for PCL scaffold in improving disease-specific survival outcomes.

As described herein, a micro-porous PCL scaffold has been developed, implanted prior to tumor initiation, and recruited metastatic cells at an early time-point in disease progression. The approach to this work was based on recapitulating some of the immunological aspects of the pre-metastatic niche, while prior reports have focused on materials to mimic properties of target organs (e.g., bone, bone marrow). Previous studies of the pre-metastatic niche have identified some of the biological cues involved in cancer cell recruitment, such as the cellular components (e.g., hematopoietic and endothelial progenitor cells, immune cells), soluble factors (e.g., cytokines, chemokines), and extracellular matrix proteins. Importantly, as indicated by Lyden, the existence of the pre-metastatic niche implies that metastasis to a particular site is not random, but is predetermined, which supports the idea that a site could be engineered to attract metastatic cells. The synthetic scaffolds herein provide an opportunity to create a defined environment with which to investigate the role of specific components involved in the colonization of metastatic cells. Scaffolds can be modified with specific niche components, such as stromal cells, VEGFR1⁺ cells, myeloid derived suppressor cells (MDSCs), ECM molecules (e.g., fibronectin, myeloperoxidase, collagen IV), cytoplasmic proteins (e.g., S100A8 and S100A9), and cytokines (e.g., IL-10, MCP-1, Haptoglobin) to identify the key signals in the metastatic environment, thereby providing a tool with which to advance fundamental studies of the pre-metastatic niche and tumor metastasis. Herein, the scaffold defines a site for immune cell infiltration, and we characterize the dynamic immune response associated with cancer cell recruitment.

The immune cell populations at the PCL scaffold, which had stabilized prior to tumor inoculation, were substantially altered post-tumor inoculation, suggesting that the changing foreign body response to the implant may contribute to metastatic cell recruitment. Immune cells are recognized as significant to the pre-metastatic niche. As such, chemokine CCL-2 recruits inflammatory monocytes (Ly6C⁺F4/80⁻ cells) to the pre-metastatic niche enabling metastasis of breast cancer cells. Similarly, CD11b⁺Gr-1^{hi}Ly6C⁻ cells are recruited via inflammatory chemoattractants (e.g., S100A8 and S100A9) to pre-metastatic niches. In addition, CD11b⁺Gr-1^{hi}Ly6C⁻ cells are known to downregulate infiltration and suppress the function of T cells (CD4⁺ and CD8⁺ T cells) and NK cells. Consistent with these observations, an increase in the levels of monocytes and CD11b⁺Gr-1^{hi}Ly6C⁻ cells was found at the scaffold site post tumor inoculation and an associated decrease in the abundance of CD4⁺ T cells, CD8⁺ T cells, and CD49b⁺ NK cells, with the greatest change observed for CD11b⁺Gr-1^{hi}Ly6C⁻ cells (i.e., more than two orders of magnitude). Importantly, the changing immune composition as a consequence of disease progression observed in the spleen largely reflected the dynamics at the scaffold site. Taken together, these results suggest that engineering a local microenvironment may be used to identify and modulate key components of cancer-associated immunogenicity in the pre-metastatic niche.

The implantation of PCL scaffolds enhanced disease-specific survival. The implantation of PCL scaffolds in the subcutaneous space reduced tumor burden in major organ sites (i.e., liver and brain) in an immunocompetent mouse model. As described in U.S. application Ser. No. 13/838,800, a reduction in burden in the lung in an immunocompromised mouse model occurred using PLG scaffolds implanted in the intraperitoneal fat pad after tumor inoculation. The present techniques, however, extend beyond these prior techniques and importantly demonstrate a scaffold-based approach that can contribute to the reduction in disease burden in solid organs in both immunocompetent and compromised mouse models and when implanted at different sites. Metastatic cells could be detected within chronically implanted PCL scaffolds by day 5, for example, following tumor inoculation using ISOCT imaging, which allowed for label free detection of metastasis through changes in the tissue ultrastructure (e.g., matrix organization) and the presence of cancer cells that have a distinct nano-scale signature relative to normal cells.

The great stability of the present techniques allows for clinically-significant increases in survival times for subjects.

The subsequent resection of the primary tumor at day 10 post tumor inoculation resulted in increased survival in mice that received a scaffold. The increased survival may result from a decreased burden of CD11b⁺Gr-1^{hi}Ly6C⁻ cells observed locally at the primary tumor and systemically in the spleen of a scaffold-bearing mouse when compared to a mouse that received a mock surgery. As stated, CD11b⁺Gr-1^{hi}Ly6C⁻ cells have been implicated in the pre-metastatic niche and the reduced abundance of these cells systemically may contribute to the reduced burden in solid organs. Finally, MDSCs have been identified in high numbers in patients with metastatic disease, correlating with clinical stage and metastatic disease burden and their levels are predictive of overall survival. Thus, a scaffold-based approach that reduces the abundance of MDSCs could, in part, explain the survival benefit observed in our studies. Taken together, the ability to detect metastatic disease at an early stage, in combination with the survival benefit provided by the scaffold highlight the potential for this technology in transforming the current detection and management of metastatic disease.

The recruitment of metastatic cells to the scaffold, combined with label-free imaging for detection of nascent stage metastatic cells, and reduced burden of disease in solid organs (i.e., liver and brain), ultimately allows for interventions when the disease burden is low that could translate to improved disease-specific outcomes. The results disclosed herein show that a scaffold for capture and detection of early metastatic cells, combined with an intervention shortly after detection of early metastasis (i.e., primary tumor excision) can actually enhance subject survival. This biomaterial approach is based on the host response to an implanted scaffold, thereby avoiding the presence of potentially deleterious cellular or biological components.

PCL is FDA approved for applications such as drug delivery, suture material, and wound dressings, which may facilitate translation to the clinical settings through existing implantable structures. Furthermore, this material is biodegradable and would not need to be retrieved unless cancer cells are detected; and the degradation rate is relatively slow allowing the implant to be monitored for up to two years within a patient. Finally, the scaffold may be integrated into current breast cancer disease management plans by potentially serving as a sentinel site for disease recurrence or could be implanted prophylactically to detect metastasis in high-risk patients and hold promise for reducing breast cancer mortality.

### PEG Scaffold

PEG gels were fabricated as described herein and were implanted at day 0 into the dorsal subcutaneous space of eight week old BALB/c mice. 2E6 4T1-tdtom-luc2 cells were injected into the fourth right mammary fat pads on day 28 post-scaffold implantation. Scaffolds were retrieved on day 42 and analyzed for the presence of tumor cells via flow cytometry.

Table 3 shows the number of cancer cells identified in scaffolds retrieved from five different mice.

TABLE 3

| Mouse Number | Scaffold | Number of cancer cells |
|---|---|---|
| M4L | PEG | 38 |
| M4R | PEG | 29 |
| M5L | PEG | 23 |
| M6L | PEG | 73 |
| M6R | PEG | 38 |

The results of the experiment show that the PEG scaffolds were able to successfully capture cancer cells in vivo.

As used herein, the term “subject” refers to any human or animal (e.g., non-human primate, rodent, feline, canine, bovine, porcine, equine, etc.).

As used herein, the term “subject suspected of having cancer” refers to a subject that presents one or more symptoms indicative of a cancer or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “initial diagnosis” refers to results of initial cancer diagnosis (e.g., the presence or absence of cancerous cells). An initial diagnosis does not include information about the stage of the cancer or the presence of metastasis.

As used herein, the term “subject at risk for cancer” refers to a subject with one or more risk factors for developing a specific cancer. Risk factors may include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, metastasis of the cancer, and the subject's prognosis.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein the term “biodegradable” refers to a material (e.g., polymer) that breaks down into smaller or component parts (e.g., oligomeric and/or monomeric units) over a period of time (e.g., typically hours to months to years) when placed (e.g., implanted or injected) into a biological environment (e.g., into the body of a subject).

As used herein, the term “resorbable” refers to a material (e.g., polymer), the degradative products of which are metabolized within or excreted from a biological environment (e.g., into the body of a subject) within which they are placed, via natural pathways.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed is:

1. A biomaterial implant comprising a micro-porous scaffold comprising poly(ε-caprolactone) (PCL) or poly(ethylene glycol) (PEG) and configured to recruit circulating metastatic cells.

2. The biomaterial implant of claim 1, wherein the scaffold comprises PCL (PCL scaffold) or PEG (PEG scaffold) and is characterized by a degradation profile that is a percent degradation over time, and wherein the scaffold has a degradation profile value of less than 50% degradation over 90 days.

3. The biomaterial implant of claim 1, wherein the PCL or PEG scaffold has a degradation profile value that is less than 25% degradation over 90 days.

4. The biomaterial implant of claim 1, wherein the PCL or PEG scaffold has a degradation profile value that is less than 10% degradation over 90 days.

5. The biomaterial implant of claim 1, wherein the PCL or PEG scaffold has a degradation profile value that is less than 5% degradation over 90 days.

6. The biomaterial implant of claim 1, wherein the PCL or PEG scaffold has a degradation profile value that is less than 1% degradation over 90 days.

7. The biomaterial implant of claim 1, wherein the scaffold comprises PEG (PEG scaffold) and is non-biodegradable and is non-resorbable.

8. The biomaterial implant of claim 7, wherein the PEG scaffold is crosslinked with a peptide or polysaccharide that is not degraded by a mammalian enzyme.

10. The biomaterial implant of claim 1 having an average mesh size of about 20 nanometers (nm) to about 50 nm.

11. The biomaterial implant of claim 1, wherein the scaffold is functionalized with at least one of a stromal cell, an extracellular matrix molecule, or a cytokine.

12. The biomaterial implant of claim 1, wherein the PEG has an average molecular weight of at least 10,000 daltons.

13. The biomaterial implant of claim 1, wherein the PEG has an average molecular weight of at least 15,000 daltons.

14. The biomaterial implant of claim 1, wherein the PEG has an average molecular weight between about 10,000 and about 20,000 daltons.

15. A biomaterial implant comprising a micro-porous scaffold comprising a non-biodegradable polymer configured to recruit circulating metastatic cells and functionalized to release the recruited circulating metastatic cells in response to engagement of an external enzyme.

16. A biomaterial implant comprising a micro-porous scaffold comprising a non-biodegradable polymer configured to recruit circulating metastatic cells and functionalized to degrade in response to engagement of an external enzyme to release the recruited circulating metastatic cells.

17. A method of capturing a metastatic tumor cell comprising implanting the biomaterial implant of any one of claims 1-16 into a subject.

18. The method of claim 17 wherein the subject suffers from cancer that has been diagnosed as metastatic.

19. The method of claim 17 wherein the subject suffers from cancer that has not been diagnosed as metastatic.

20. The method of any one of claims 17-19 wherein the implanting is subcutaneous or intramuscular.

21. The method of any one of claims 17-20 wherein the implanting occurs at one site in the subject.

22. The method of any one of claims 17-20 wherein the implanting occurs at more than one site in the subject.

23. The method of any one of claims 17-21 wherein one biomaterial implant is implanted.

24. The method of any one of claims 17-22 wherein more than one biomaterial implant is implanted.

25. The method of any one of claims 21-24 wherein the site is the lung, liver, brain, bone, peritoneum, omental fat, muscle, or lymph node.

26. The method of any one of claims 17-25 further comprising removing the biomaterial implant or implants.

27. The method of claim 26 further comprising detecting a metastatic cell, the detecting comprising one or more of inverse-scattering optical coherence tomography (ISOCT), fluorescence activated cell sorting (FACS), high frequency ultrasound, ultrasound, positron emission tomography (PET) scan, magnetic resonance imaging (MRI), photoacoustic imaging, or fluorescence imaging.

28. The method of any one of claims 17-27 wherein the capturing lowers tumor burden of the subject.

29. The method of any one of claims 17-28 further comprising administering to the subject a chemotherapeutic agent.

30. The method of any one of claims 18-29 further comprising surgically removing the cancer from the subject.

31. The method of any one of claims 17-30 further comprising administering radiotherapy to the subject.

32. The method of any one of claims 26-31, further comprising retrieving the captured metastatic tumor cell from the scaffold.

33. The method of any one of claims 26-32 further comprising retrieving a captured non-tumor cell from the scaffold.

34. The method of any one of claims 17-33 wherein survival rate of the subject is increased relative to a subject in whom the biomaterial implant was not implanted.

35. A method of analyzing effectiveness of a treatment to reduce metastasis in a subject comprising:
(i) implanting at least a first and a second biomaterial implant into the subject and maintaining for a period of time wherein each implant is according to any one of claims 1-16;
(ii) removing the first biomaterial implant and determining a first amount of metastasis;
(iii) administering the treatment to the subject;
(iv) removing the second biomaterial implant and determining a second amount of metastasis;
(v) wherein the treatment is effective to reduce metastasis if the second amount of metastasis is lower than the first amount of metastasis.

36. The method of claim 35 wherein the first amount of metastasis and the second amount of metastasis are determined by one or more of inverse-scattering optical coherence tomography (ISOCT), fluorescence activated cell sorting (FACS), high frequency ultrasound, ultrasound, positron emission tomography (PET) scan, magnetic resonance imaging (MRI), photoacoustic imaging, or fluorescence imaging.

37. The method of claim 35 or 36 wherein the period of time is about two years.
